(19) United States
(12) Patent Application Publication
(10) Pub. No.: US 2018/0008290 A1
(43) Pub. Date: Jan. 11, 2018

(54) LASER GUIDANCE SYSTEM FOR INTRA-OPERATIVE ORTHOPEDIC SURGERY

(71) Applicant: Whale Imaging, Inc., Waltham, MA (US)

(72) Inventors: Changguo Ji, Lexington, MA (US); Xinbai He, Belmont, MA (US); Xun Zhu, Waltham, MA (US)

(21) Appl. No.: 15/631,208

(22) Filed: Jun. 23, 2017

Related U.S. Application Data
(60) Provisional application No. 62/358,759, filed on Jul. 6, 2016.

(51) Int. Cl. A61B 17/17; A61B 5/06; A61B 5/00; A61B 6/00

(52) U.S. Cl. CPC A61B 17/1703; A61B 6/547; A61B 5/061; A61B 5/0059; A61B 2034/107

(57) ABSTRACT

A system and method are provided for performing fluoroscopic procedures with assistance of guiding laser beam projections to reduce a reliance on harmful radiation emitting fluoroscopic imaging devices during the procedure. The system and method reduce an amount of radiation exposure to patients and medical personnel during procedures that require assistive real-time imaging. Specifically, an automated laser guidance system and method of use is provided to reduce fluoroscopic radiation, reduce operation time, and increase operative accuracy.

### CROSS-REFERENCE TO RELATED APPLICATION(S)

This application claims priority to, and the benefit of, co-pending U.S. Provisional Application No. 62/358,759, filed Jul. 6, 2016, for all subject matter common to both applications. The disclosure of said provisional application is hereby incorporated by reference in its entirety.

### FIELD OF THE INVENTION

The present invention relates to a laser guidance system for use in intra-operative orthopedic surgery or fluoroscopy. In particular, the present invention relates to a system and corresponding method of use implementing a diode array configured to create an improved laser guidance system for use with a fluoroscopic medical imaging system to produce guiding laser beam projections on a patient to assist a surgeon in a real-time fluoroscopic imaging during procedures.

### BACKGROUND

Generally, fluoroscopy is considered indispensable during orthopedic surgery. However, there is increasing concern regarding occupational safety in the operating room (OR). Orthopedic surgeons are increasingly using X-ray based fluoroscopic techniques in the operation theatre or in the fluoroscopy room. Procedures such as kyphoplasty, vertebroplasty, deformity correction, pelvic fixation, intramedullary inter-locking nails and computerized tomography (CT) guided biopsies require radiation exposure. Vertebroplasty and kyphoplasty are similar medical spinal procedures. Such procedures rely on X-ray based fluoroscopic techniques to improve surgery success and efficiency. For example, these spinal procedures include methodologies in which bone cement is injected through a small hole in the skin percutaneously into a fractured vertebra with the goal of relieving back pain caused by vertebral compression fractures. In practice, using an X-ray, a surgeon inserts a dedicated guide wire to confirm position under image intensification multiple times before drilling and tapping. Utilization of such processes can inevitably introduce additional radiation doses to both surgeons and patients.

The general philosophy followed by most healthcare facilities is to minimize radiation dosages and require all radiation exposures to be justified. In particular, industry practice is to keep exposure levels “as low as reasonably achievable” (ALARA). Even in following ALARA, there is increasing concern regarding occupational safety in the operating room (OR). In particular, during a course of a career, an orthopedic surgeon and their OR staff could be exposed to potentially dangerous cumulative levels of radiation. This long term exposure can cause substantial cytogenetic and chromosomal damage, potentially increasing cancer risk. Even relatively small doses of radiation should be considered dangerous over the long-term. Additionally, protective measures, including observance of safe working distance from the radiation source and the routine use of protective garments, have been established.

### SUMMARY

There is a need for improved methods and systems to reduce fluoroscopic radiation exposure to patients, reduce procedure time, and increase operative accuracy. The present invention is directed toward further solutions to address this need, in addition to having other desirable characteristics. Specifically, a medical imaging and guidance system is provided to produce guiding laser beam projections on a patient to assist a surgeon in a fluoroscopic imaging procedure in lieu of active imaging processes (e.g., X-ray). The guiding laser beam projections are generated by a fixed array of laser diodes and through their use, less radiation-based imaging is required during an operation, therefore reducing radiation exposure time and increasing operative accuracy over alternative non-fluoroscopic methodologies. In particular, the present invention provides a system and method for a laser guidance system to be used in connection with intra-operative fluoroscopic imaging, but in a manner that reduces activation periods of the imaging devices because laser diode generated guiding laser beam projections are used to facilitate positioning on the patient's body.

In accordance with example embodiments of the present invention, a medical imaging and guidance system is provided. The system includes a fluoroscopic imaging system. The fluoroscopic imaging system includes a support gantry having a generally arc shape about an interior center focus point with a first terminal end and a second terminal end. The fluoroscopic imaging system also includes a first imaging assembly that is positioned on the support gantry and comprising a first imaging energy emitter that is positioned opposite a first imaging receptor, wherein one of the first imaging energy emitter or the first imaging receptor is positioned at the first terminal end of the support gantry. The fluoroscopic imaging system further includes a plurality of laser-diodes fixedly attached to at least one of the first imaging receptor or the second imaging receptor around half of a circumference of the first imaging receptor or the second imaging receptor, respectively, at intervals of no greater than 15 radial degrees of spacing between each laser-diode of the plurality of laser-diodes. The plurality of laser-diodes emits guiding laser beam projections onto a subject to provide guidance to medical personnel during a procedure without radiation energy being emitted by the first imaging assembly.

In accordance with aspects of the present invention, the fluoroscopic imaging system further includes a second imaging assembly that is positioned on the support gantry and comprising a second imaging energy emitter positioned that is opposite a second imaging receptor, wherein one of the second imaging energy emitter or the second imaging receptor is positioned at the second terminal end of the support gantry and a control unit that directs movement and positioning of the support gantry. The s fluoroscopic imaging system can further include a processing and display device in communication with the first imaging assembly, the second imaging assembly, and the plurality of laser-diodes. The fluoroscopic imaging system obtains raw image data of a subject patient located proximate the interior center focus point between the first imaging assembly and the second imaging assembly and communicates the raw image data to the processing and display device. The processing and display device receives the raw image data and transforms the raw image data for display as a preview image and receives at least one plan line that is overlaid onto the preview image and electronically selects one or more of the plurality of laser-diodes to activate to project one or more guiding laser beam projections onto the subject patient at locations corresponding to the at least one plan line. The plurality of laser-diodes can be mechanically positioned with an angular coverage or linear translation around the fluoroscopic imaging system. The at least one plan line can be generated with any position and line direction through the preview image. The at least one plan line can provide input information and imaging geometry to determine power state of each of the plurality of laser-diodes. The first imaging assembly can be positioned and oriented to emit imaging energy in an LT plane and the second imaging assembly is positioned and oriented to emit imaging energy in an AP plane, perpendicular to the LT plane. The first imaging assembly can be positioned and oriented to emit imaging energy in an AP plane and the second imaging assembly is positioned and oriented to emit imaging energy in an LT plane, perpendicular to the AP plane. The first imaging receptor and the second imaging receptor can be one of an image intensifier, a flat panel detector, or a thin film transistor (TFT) flat-panel detector with a scintillation material layer configured to readout a voltage data value to the processing and display device. The TFT flat-panel detector can be configured to receive energy from visible photons that charge capacitors of pixel cells within the TFT flat-panel detector and charges from each of the pixel cells are readout as a voltage data value to the processing and display device. The first imaging energy emitter and the second imaging energy emitter can be X-ray sources configured to produce X-ray beams.

In accordance with aspects of the present invention, the plurality of laser-diodes is mechanically aligned to produce guiding laser beam projections to pass through the interior center focus point of the support gantry. The plurality of laser-diodes further can include at least three laser-diodes uniformly spaced around the half of the circumference of the first imaging receptor or the second imaging receptor. An angle of convergence for each the plurality of laser-diodes can be provided to focus each of the plurality of laser-diodes to a center of a front input plane of an imaging receptor that the plurality of laser-diodes is attached thereto. Each of the plurality of laser-diodes can be independently operable according to a user specification.

In accordance with example embodiments of the present invention, a method for utilization of a medical procedure guidance system is provided. The method includes activating an imaging device including a first imaging assembly and a second imaging assembly configured and arranged to receive a subject patient therebetween. The method also includes obtaining, by imaging receptors, a raw image data of the subject patient, communicating the raw image data to a processing and display device, and transforming the raw image data, by the processing and display device, into a preview image of the subject patient. The method further includes displaying the preview image on a display with at least one plan line, generated by the processing and display device, overlaid onto the preview image and instructing, by the processing and display device, one or more laser-diode of a plurality of laser-diodes in a laser-diode array to project one or more guiding laser beam projections onto the subject patient at locations corresponding to the at least one plan line, which is overlaid onto the preview image.

In accordance with aspects of the present invention, the at least one plan line is generated in response to receiving user input to create the at least one plan line at a particular orientation and location on the preview image of the subject patient.

In accordance with aspects of the present invention, the method further includes determining a positioning of the one or more laser-diode of the plurality of laser-diodes to generate the one or more guiding laser beam projections. The method can also include performing a fluoroscopic procedure relying on the one or more guiding laser beam projections.

In accordance with aspects of the present invention, the imaging device further includes a first imaging energy emitter, which is positioned opposite a first imaging receptor. One of the first imaging energy emitter or the first imaging receptor is positioned at a first terminal end of a support gantry. The second imaging assembly is positioned on the support gantry, the second imaging assembly including a second imaging energy emitter positioned opposite a second imaging receptor. One of the second imaging energy emitter or the second imaging receptor is positioned at a second terminal end of the support gantry. The imaging device also includes a control unit that directs movement and positioning of the support gantry and the plurality of laser-diodes are fixedly attached to at least one of the first imaging receptor or the second imaging receptor around half of a circumference of the first imaging receptor or the second imaging receptor.

### BRIEF DESCRIPTION OF THE FIGURES

These and other characteristics of the present invention will be more fully understood by reference to the following detailed description in conjunction with the attached drawings, in which:

FIG. 1 is diagrammatic illustration of a conventional fluoroscopic imaging device known in the art;

FIG. 2 is an illustrative example of a conventional two-diode laser guidance system known in the art for use with a fluoroscopic imaging device;

FIG. 3 is diagrammatic illustration of a fluoroscopic imaging device in accordance with the present invention;

FIG. 4 is an illustrative example configuration of a plurality of laser diodes for implementation on an imaging device in accordance with the present invention;

FIG. 5A is a fluoroscopic image with a plan line, in accordance with aspects of the present invention;

FIG. 5B is an image of guiding laser beam projections generated by the laser diodes in accordance with the present invention;

FIGS. 6A and 6B are illustrative examples of guiding laser beam projections generated by the laser diodes in accordance with the present invention;

FIG. 7 is a flowchart depicting a method of utilizing the laser diode array laser guidance system in accordance with the present invention; and

FIG. 8 is a diagrammatic illustration of a high level architecture for implementing processes in accordance with aspects of the invention.

### DETAILED DESCRIPTION

An illustrative embodiment of the present invention relates to a fluoroscopic imaging device configured with a plurality of laser diodes, or laser diode array, installed on at least one of the imaging receptors of the fluoroscopic imaging device. The plurality of laser diodes is distributed in a semi-circular pattern on the imaging receptor(s) at stationary locations. The plurality of laser diodes is utilized to create a guiding laser beam projections on a subject for use during a fluoroscopic procedure. A surgeon can rely upon the guiding laser beam projections produced by the plurality of laser diodes instead of requiring an imaging device to be active for the entirety of the fluoroscopic procedure (unnecessarily exposing the surgeon, patient, and staff to radiation). In other words, the guiding laser beam projections provide guidance during the procedure in lieu of an active imaging device (e.g., X-ray).

To generate the guiding laser beam projections, one or more of the plurality of laser diodes is activated based on a positioning and orientation of a user-provided plan line input that is overlaid on a fluoroscopic preview image. Upon activation, the one or more laser diodes produce the guiding laser beam projections on a subject which correspond to the location and orientation of the user-provided plan line. The plurality of laser diodes are spaced, oriented, and positioned in a manner to provide 360 degree coverage of guiding laser beam projections to facilitate coverage of all requisite angels and areas, and enable production of a guiding laser beam projections matching any provided plan line. The use of laser diodes generating guiding laser beam projections directly on the patient and corresponding to plan lines in a fluoroscopic image make it possible for the fluoroscopic imaging radiation to be shut off and still have guiding laser beam projections displayed to the medical professionals to rely upon during a procedure.

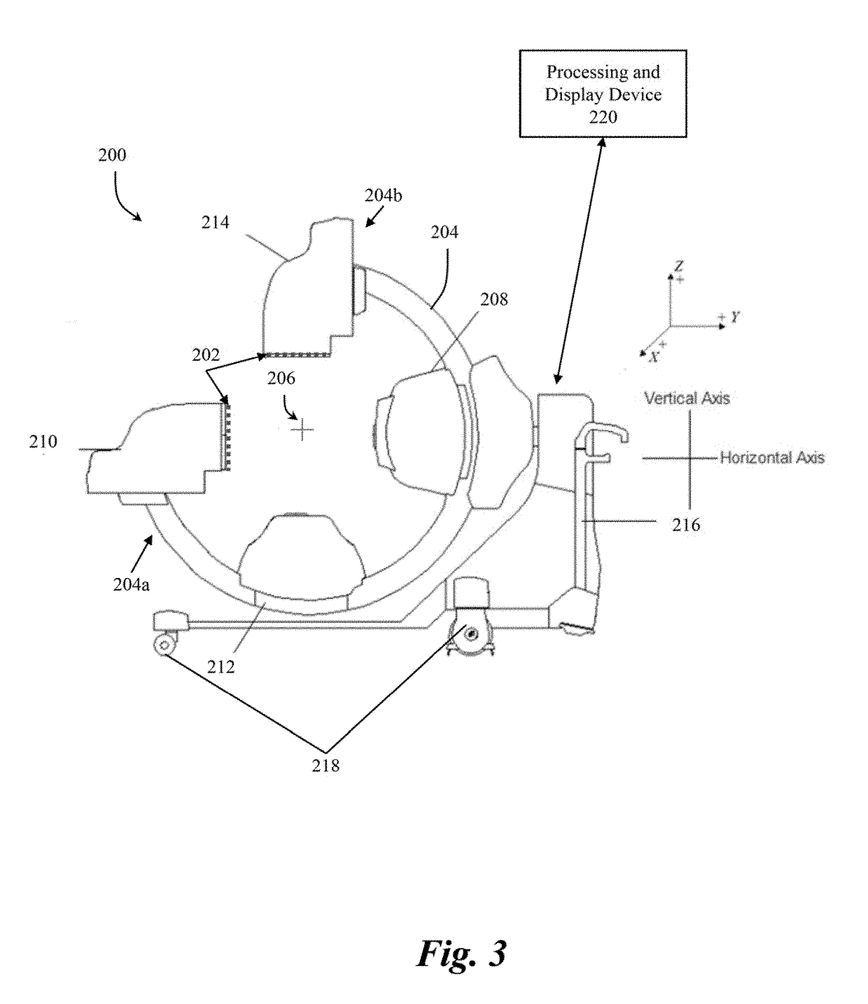

FIGS. 3 through 8, wherein like parts are designated by like reference numerals throughout, illustrate an example embodiment or embodiments of improved operation for the medical imaging and laser guidance system, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

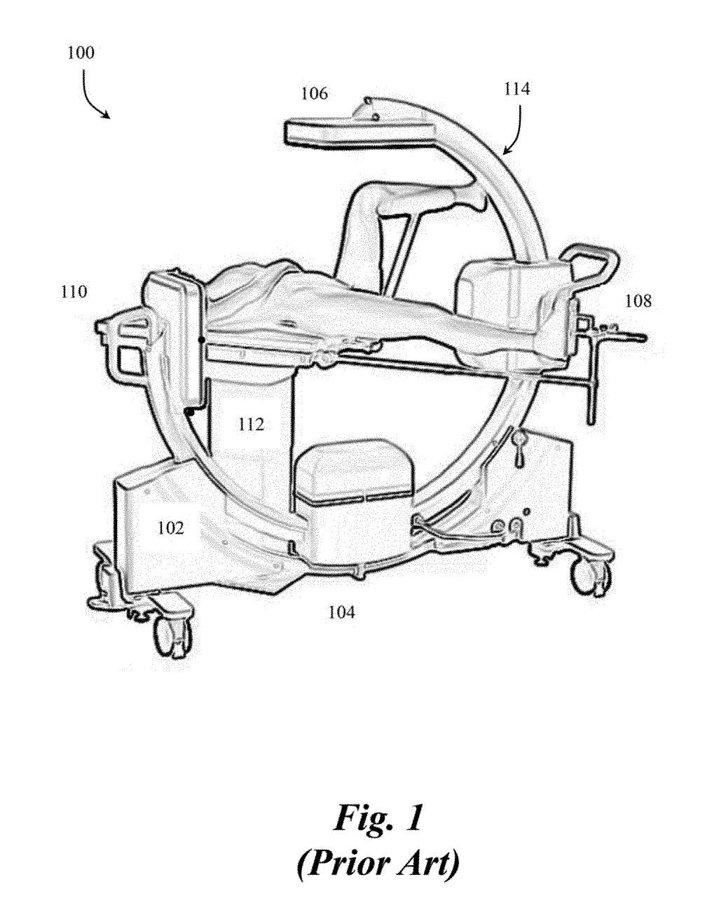

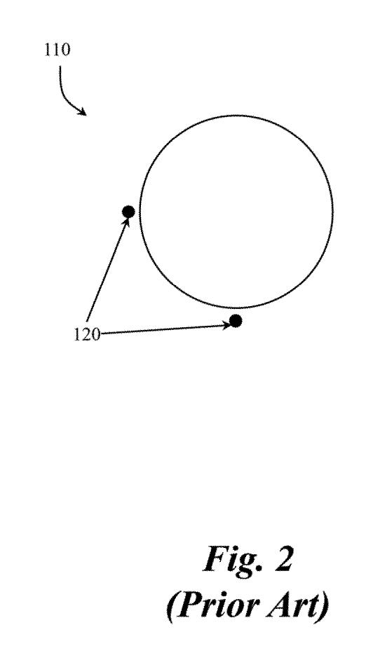

Imaging systems are commonly utilized in the medical field for use during fluoroscopic procedures and come in a variety of configurations for a variety of applications (e.g., C-arm single plane imager, G-arm bi-plane imager, etc.). An example of an imaging system configured for capturing bi-planar medical images (e.g., X-rays) of a patient is depicted in FIGS. 1 and 2. In particular, FIG. 1 depicts a conventional G-arm medical imaging system 100 and the main components that make up the G-arm medical imaging system 100. The main components of the G-arm medical imaging system 100 system include a movable stand 102, a imaging energy emitter 104 (e.g., an X-ray source, X-ray tube, etc.) and imaging receptor 106 (e.g., an image intensifier, flat panel detector, etc.) configured for a frontal view (or anteroposterior view), an imaging energy emitter 108 and imaging receptor 110 configured for a lateral view, and a patient table 112 configured to hold a patient between the imaging energy emitters 104, 108 and the imaging receptor 106, 110. As would be appreciated by one skilled in the art, the imaging energy emitters 104, 108 can include any kind of suitable imaging energy emitters utilized for imaging a patient. For example, the imaging energy emitters 104, 108 can be electromagnetic radiation or x-imaging energy emitters configured to produce X-rays. The combination of elements in the G-arm medical imaging system 100 includes a gantry 114 that supports all of the components/machinery. The gantry 114 of the G-arm medical imaging system 100 is configured to enable two bi-planar images to be captured simultaneously or without movement of the equipment and/or the patient. In some instances, the gantry 114 is adjustable to change angles of the imaging machinery (e.g., the imaging energy emitters 104, 108 and imaging receptor 106, 110).

FIG. 2 depicts a conventional imaging receptor 110 (or imaging receptor 106) with two laser diodes 120 installed thereon. The laser diodes 120 are installed on the imaging receptor 110 in a configuration in which the diodes can rotate around a circular position on the imaging receptor surface. Additionally, the laser diodes 120 are configured to project a laser guide beam on a surface of subject (e.g., patient) positioned within the G-arm medical imaging system 100. An example of such a laser guidance system and methodology is discussed with respect to U.S. patent application Ser. No. 15/426,791, incorporated herein by reference. In short, a user can lay out a plan line or guiding laser beam on an image preview (e.g., an X-ray) to be projected on the patient as a guiding laser beam projections to assist in performing a fluoroscopic image supported surgical or medical procedure. Based on the plan line laid out by the user, the G-arm medical imaging system 100 instructs the laser diodes 120 to position and rotate in a configuration to project the guiding laser beam projections on the subject at a location corresponding to the plan line on the preview image.

Continuing with FIG. 2, the laser diodes 120 are positioned at two separate locations separated by 90 degrees on the imaging receptor 110. In particular, the laser diodes 120 are positioned at the six and nine o'clock positions of a circular area within the imaging receptor 110. As would be appreciated by one skilled in the art, the laser diodes 120 can be positioned at any locations on an imaging receptor that enables them to be rotated and positioned in a manner to create a guiding laser beam projection on a subject at various locations and orientations. However, the utilization of two laser diodes adds complexity to the imaging receptor 110 and adds configuration time during a procedure. In particular, the utilizing of two laser diodes requires mechanisms to enable the ability to rotate and position the diodes in a position necessary to provide the laser beam projections. Additionally, the rotation and positioning takes additional time during a procedure whenever the diodes need to be repositioned for creating the guiding laser beam projection. As such, the two diode configuration is more complex and more time consuming to operate.

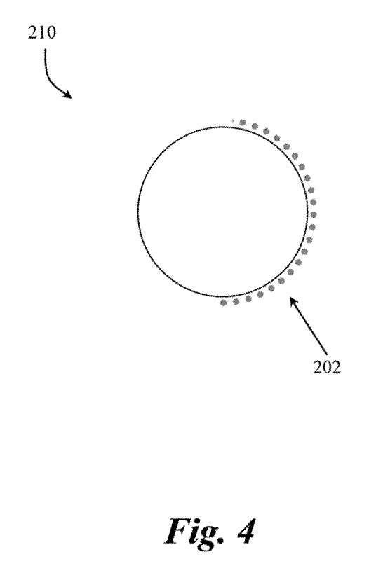

FIG. 3 depicts example illustrations of a medical imaging and guidance system including an imaging apparatus 200 for use in accordance with the present invention. The apparatus 200 of the present invention shares similar components and functionality with the components discussed with respect to the G-arm medical imaging system 100 in FIG. 1. The apparatus 200 also includes additional components and functionality in combination with the traditional components of the G-arm medical imaging system 100. In particular, FIG. 3 depicts an imaging apparatus 200 configured with a plurality of laser diodes 202, or diode array, including a plurality of diodes 202 configured to generate one or more guiding laser beam projections on a target location (e.g., a patient). In accordance with an example embodiment of the present invention, the plurality of laser diodes 202 are configured at fixed locations on one of the image receptors (210, 214) in a semi-circular pattern, as depicted in FIG. 4.

The apparatus 200 includes a support gantry 204 having a generally arc shape, about an interior center focus point 206, with a first terminal end 204a and a second terminal end 204b. The apparatus 200 also includes a first imaging assembly positioned on the support gantry 204, the first imaging assembly includes a first imaging energy emitter 208 (e.g., X-ray source) positioned opposite a first imaging receptor 210. As would be appreciated by one skilled in the art, the first imaging receptor 210 is configured to receive the energy format produced by the first imaging energy emitter 208. For example, the first imaging energy emitter 208 is an X-ray source and the first imaging receptor 210 is a flat-panel detector configured to receive X-ray energy. One of the first imaging energy emitter 208 and the first imaging receptor 210 is located proximate the first terminal end 204a.

In accordance with an example embodiment of the present invention, as depicted in FIG. 3, the first imaging receptor 210 is located at the first terminal end 204a of the support gantry 204. As would be appreciated by one skilled in the art, the first imaging energy emitter 208 could be positioned at the first terminal end 204a with the first imaging receptor 210 positioned on the opposite side of the support gantry 204 without influencing the imaging process. In other words, the first imaging receptor 210 (shown in FIG. 3) can be swapped with the first imaging energy emitter 208 positionally (shown in FIG. 3) and be an equivalent configuration. The first imaging assembly is positioned and oriented to emit imaging energy (e.g., from the first imaging energy emitter 208) in an LT plane, as depicted in FIG. 3. Additionally, as would be appreciated by one skilled in the art, the first imaging assembly can alternatively be positioned and oriented to emit imaging energy (e.g., from the energy emitter 212) in an AP plane.

Continuing with FIG. 3, the apparatus 200 further includes a second imaging assembly positioned on the support gantry 204; the second imaging assembly including a second imaging energy emitter 212 (e.g., X-ray source) positioned opposite a second imaging receptor 214. As would be appreciated by one skilled in the art, the second imaging receptor 214 is configured to receive the energy format produced by the second imaging energy emitter 212. For example, the second imaging energy emitter 212 is an X-ray source and the second imaging receptor 214 is a flat-panel detector configured to read X-ray energy. One of the second imaging energy emitter 212 or the second imaging receptor 214 is positioned at the second terminal end 204b of the support gantry 204. In accordance with an example embodiment of the present invention, as depicted in FIG. 3, the second imaging receptor 214 is positioned proximate to the second terminal end 204b of the support gantry 204. As would be appreciated by one skilled in the art, the second imaging energy emitter 212 could be positioned at the second terminal end 204b with the second imaging receptor 214 positioned on the opposite side of the support gantry 204 without influencing the imaging process. In other words, the second imaging receptor 214 (shown in FIG. 3) can also be switched with the second imaging energy emitter 212 (shown in FIG. 3) in an optional arrangement. The second imaging assembly is positioned and oriented to emit imaging energy in an AP plane, perpendicular to the LT plane created by the first imaging assembly, as depicted in FIG. 3. Additionally, as would be appreciated by one skilled in the art, the second imaging assembly can be positioned and oriented to emit imaging energy in an LT plane, perpendicular to the AP plane from the first imaging assembly.

The apparatus 200 also includes a control unit 216 configured to move and position the support gantry 204 at a desired location. Additionally, the support gantry 204 includes a plurality of wheels 218 to enable a user to push, pull, and pivot the apparatus 200 into a desired position via the control unit 216. In accordance with an example embodiment of the present invention, the apparatus 200 includes or is otherwise in communication with a processing and display device 220 (such as the imaging control device discussed in U.S. Patent Application Publication No. 2016/0262712 incorporated herein by reference). The processing and display device 220 is configured to receive raw image readouts (of a subject located proximate the interior center focus point 206) from the imaging receptors 210, 214 and convert the readouts signal into a displayable format. The imaging receptors 210, 214 can include any combination of image receptors known in the art configured to provide readable signals to the processing and display device 220 for display. For example, the imaging receptors 210, 214 can be thin film transistor (TFT) panels with a scintillation material layer configured to receive energy from visible photons to charge capacitors of pixel cells within the TFT panel. The charges for each of the pixel cells are readout as a voltage data value to the processing and display device 220. The signals received by the processing and display device 220 can be configured into two dimensional or three dimensional images utilizing any combination of methodologies known in the art.

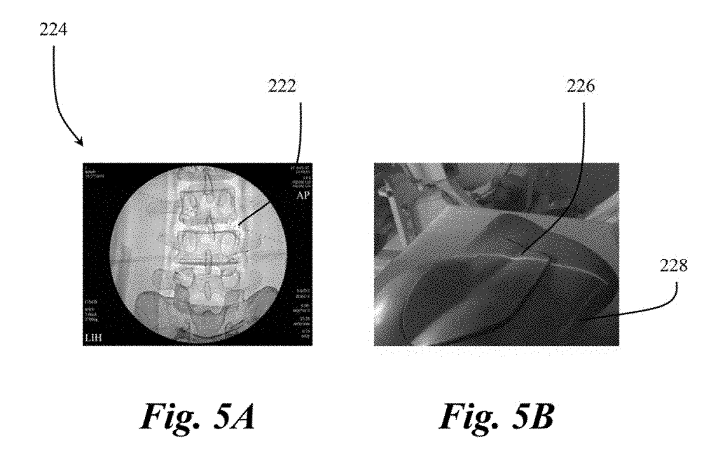

In accordance with an example embodiment of the present invention, the processing and display device 220 is configured to provide a user with a planning tool to create one or more plan lines 222 on a preview image 224 for the generation of a guiding laser beam projection 226, by one or more laser diodes 202, on a subject 228, as depicted in FIGS. 5A and 5B and discussed in greater detail with respect to U.S. patent application Ser. No. 15/426,791 (as it relates to the plan lines 222). In an example, a surgeon can create the one or more plan lines 222 on a fluoroscopic preview image 224 through a software interface provided by the processing and display device 220 by drawing the one or more plan lines 222 on the preview image 224 using a computer mouse, a touch screen input, drawing tablet, or other input device known in the art. The one or more plan lines 222 on the preview image 224 can be planned with any position and line direction that the user desires. As would be appreciated by one skilled in the art, the fluoroscopic preview image 224 is provided by the imaging provided by the apparatus 200. As shown in FIG. 5B, the guiding laser beam projection 226 by one or more laser diodes 202 is projected on a target location 228 corresponding to the same location as that of the plan lines 222 depicted in the preview image 224.

FIG. 4 depicts an example configuration of the plurality of laser diodes 202 fixedly attached to stationary positions within or on the first imaging receptor 210. The plurality of laser diodes 202 are oriented such that they generate laser beams focused to the field of view at the interior center focus point 206. As depicted in FIG. 4, in accordance with an example embodiment of the present invention, the plurality of laser diodes 202 is uniformly installed in a semi-circular pattern on the first imaging receptor 210 and/or the second imaging receptor 214. As would be appreciated by one skilled in the art, the laser diodes 202 can be adapted to fit on different shaped imaging receptors. For example, for an image intensifier imaging receptor, the plurality of laser diodes 202 can be mounted around the input window of the image intensifier. Additionally, the laser diodes 202 can be positioned and spaced in any shape and distribution which will allow guiding laser beam projections to be projected on a subject (e.g., a patient) at any position and configuration. For example, the plurality of laser diodes 202 can be spaced in a 180 degree distribution at intervals of no greater than 15 radial degrees of spacing between each laser-diode of the plurality of laser-diodes to form a semi-circular design. The 180 degree distribution enables the plurality of laser diodes 202 to create guiding laser beam projection coverage in a 360 degree radius. Furthermore, any number of laser diodes 202 can be utilized to make up the desired shape. Continuing the previous example, the plurality of laser diodes 202 can include at least three, and preferably more, laser-diodes uniformly spaced around the half of the circumference of each of the first imaging receptor 210 and/or the second imaging receptor 214. For example, the plurality of laser diodes 202 can include about twenty laser-diodes uniformly spaced around the half of the circumference of the first imaging receptor 210, as depicted in FIG. 4. As would be appreciated by one skilled in the art, the number of laser diodes 202 is restricted due to a size of the diodes and mounting spacing of the diodes around the imaging receiver.

In operation, in accordance with an example embodiment of the present invention, each of the laser diodes 202 is configured to generate a guiding laser beam projection 226 to be properly aligned to focus on the interior center focus point 206 of the apparatus 200. In particular, the plurality of laser diodes 202 are mechanically aligned to produce guiding laser beam projection 226 to pass through the interior center focus point 206 of the support gantry 204. The mechanical positioning of the plurality of laser diodes 202 provides an angular coverage or linear translation around the apparatus 200 (e.g., fluoroscopic imaging system). In particular, the plurality of laser diodes 202 are positioned at an angle of convergence to provide an angular focus directed to a center of a front input plane of the imaging receptor 210, 214 that the plurality of laser-diodes are attached thereto.

In accordance with an example embodiment of the present invention, each of the plurality of laser diodes 202 is independently operable according to a user specification. In particular, a user will specify a plan line to be generated by one or more of the plurality of laser diodes 202. For example, surgeons can plan line on a preview image, as discussed with respect to FIGS. 2, 5A, 5B, 6A, and 6C, to be generated by the plurality of laser diodes 202. The user specified plan line will dictate, via the processing and display device 220, the power state of each of the plurality of laser diodes 202 such that the one or more laser diodes that will produce a guiding laser beam projection corresponding to the plan line will be powered/activated.

The one or more plan lines 222 created on the preview image 224 are utilized by the processing and display device 220 to determine which diodes within the plurality of laser diodes 202 for display should be activated to generate the guiding laser beam projections 226 on the subject 228. As would be appreciated by one skilled in the art, the number and locations of the diodes that are activated is based on the desired guiding laser beam projections 226 (e.g., based on the one or more plan lines 222). In accordance with an example embodiment of the present invention, the one or more plan lines 222 are prescribed on the preview image 224 through a graphical user interface (GUI) and the geometrical input data from the one or more plan lines 222 is used to determine which laser diodes of the plurality of laser diodes 202 to activate. In particular, the processing and display device 220 receives the raw image data from the imaging receptors 210, 214 and transforms the raw image data for display as a preview image 224 and subsequently receives at least one plan line that is overlaid onto the preview image 224 from a user. Thereafter, the processing and display device 220 electronically selects one or more of the plurality of laser-diodes 202 to activate in order to project one or more guiding laser beam projections onto the subject patient at locations corresponding to the at least one plan line. In other words, based on the received plan line(s), the processing and display device 220 selects one or more laser diodes 202 (to provide an activation signal/instruction) at positions required to generate corresponding guiding laser beam projections 226 to be displayed onto the subject 228 in the same locations corresponding to the one or more plan lines 222 created on the preview image 224, as depicted in FIGS. 5A and 5B. In response to the activation, the activated laser diode(s) 202 produce the guiding laser beam projections 226 corresponding to the positioning of the one or more plan lines 222 on the preview image 224, while the first and/or second imaging assemblies are not actively emitting radiation and providing real-time imaging. As such there is no requirement that first and/or second imaging assemblies emit radiation and provide real-time imaging for the entirety of a procedure because at moments during the procedure the guiding laser beam projections can take the place of the necessary functionality of the plan lines of the first and/or section imaging assemblies, thereby reducing overall radiation emission and exposure to the patient and medical personnel. In accordance with an example embodiment of the present invention, the activation is performed by transmitting a power signal to the corresponding laser diode(s). This methodology is performed without requiring any movement of any of the plurality of laser diodes 202 and the surgeon can rely on the guiding laser beam projections 226 produced by the activated laser diode(s) from the plurality of laser diodes 202 during the intra-operative surgery without exposing the subject 228 to additional radiation beyond that which was experienced during the brief snapshot of the fluoroscopic preview image 224.

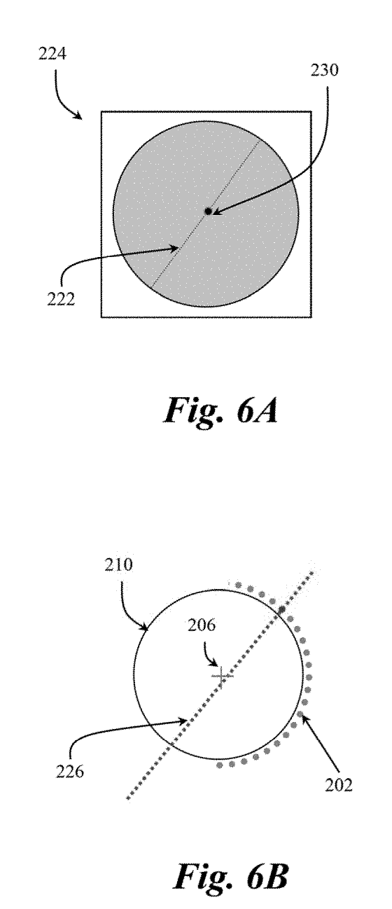

FIGS. 6A and 6B depict illustrative examples of the functionality provided by the plurality of laser diodes 202. In particular, FIG. 6A depicts a plan line 222 or surgical guide direction line, which passes through an image center 230 on preview image 224. The plan line 222 is generated with any position and line direction through the preview image 224. Additionally, the plan line 222 provides the input information and imaging geometry to determine power state of each of the plurality of laser diodes 202. In particular, the plurality of laser-diodes 202 are electronically selected according to a direction of the plan line 222 overlaid on the preview fluoroscopic image 224. FIG. 6B depicts a guiding laser beam projections 226 or surgical laser guide beam generated by the electronically selected diodes according to the plan line 222 created on the preview image 224. The guiding laser beam projection 226 generated by the activated laser diode(s) (as depicted in FIG. 6B) correspond to the plan line 222 created on the preview image 224 (as depicted in FIG. 6A).

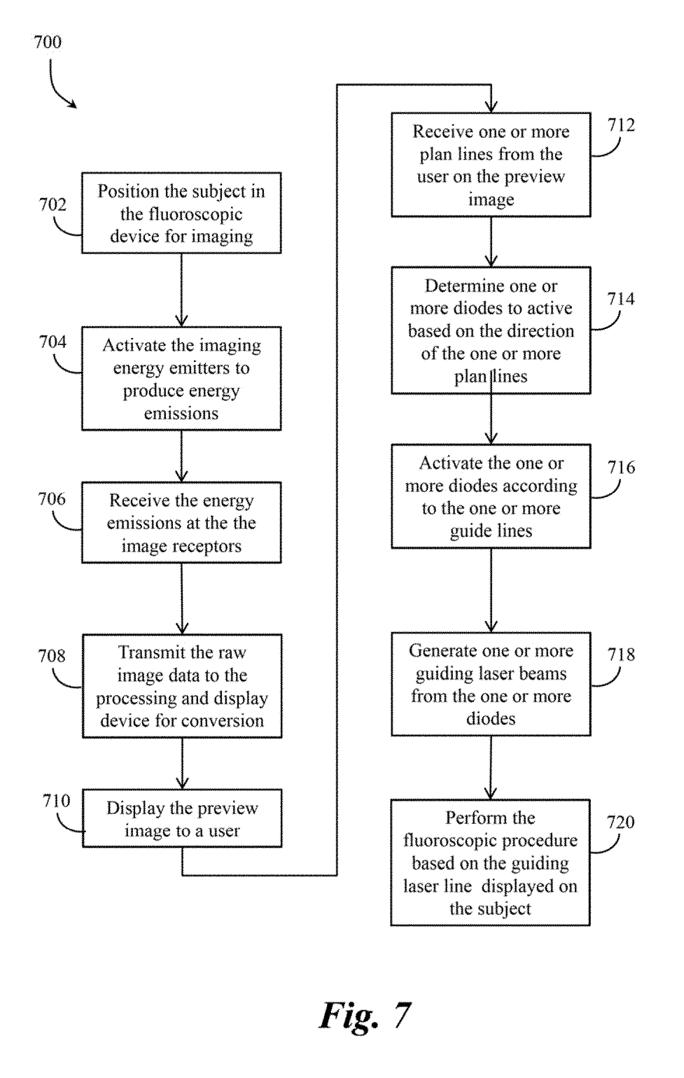

FIG. 7 depicts an example process 700 of operation for the imaging apparatus 200 discussed with respect to FIGS. 3-6B. In particular, FIG. 7 depicts an example implementation of the process 700 to pre-plan one or more plan lines 222 on a fluoroscopic preview image 224 to be directed as a guiding laser beam projections 226 (as produced by one or more of the plurality of laser diodes 202) onto a subject 228. The utilization of the guiding laser beam projections 226, as provided in process 700, enable a medical professional to perform a fluoroscopic procedure without having to constantly expose the subject and other personnel to radiation from the imaging device. In particular, the plurality of laser-diodes 202 emit guiding laser beam projections 226 onto a subject 228 to provide guidance to medical personnel during a procedure without radiation energy being emitted by either imaging assembly 208, 212. Although, with respect to FIG. 7, the process 700 is discussed with respect to the operation for the apparatus 200 depicted in FIG. 3, as would be appreciated by one skilled in the art, the process 700 could be implemented utilizing any other combination of imaging methods and systems.

At step 702 the subject 228 is positioned proximal to the interior center focus point 206 of the apparatus between the first imaging assembly and the second imaging assembly. In particular, the subject 228 is positioned in within the apparatus at a location to capture the desired area of the subject 228.

At step 704 the imaging energy emitters 208, 212 are activated. In particular, the imaging energy emitters 208, 212 (e.g., X-ray sources) are activated in response to receiving a user instruction input into the processing and display device 220 and transmitted to the x imaging energy emitters 208, 212 via the control logic. Upon activation, the imaging energy emitters 208, 212 generate energy (e.g., X-ray photons) in a direction of the subject 228 located within the apparatus 200.

At step 706 the energy emissions (e.g., X-ray photons) from the imaging energy emitters 208, 212 are absorbed as energy charges at the the imaging receptors 210, 214. In particular, the imaging receptors 210, 214 receive the energy as raw image data to be transmitted to the processing and display device 220 for conversion.

At step 708 the raw image data received by the imaging receptors 210, 214 transmitted to the processing and display device 220. Thereafter, the processing and display device 220 receives the raw image data and converts the raw image data into a format for display, utilizing any system or method known in the art. During this period of time the imaging energy emitters 208, 212 can be powered off to stop any continued radiation exposure.

At step 710 the processing and display device 220 displays a preview image 224 on a display device (e.g., a monitor) for interpretation and pre-planning by a user. The preview image 224 displayed to the user is an image of the subject 228 (e.g., X-ray image).

At step 712 the processing and display device 220 receives one or more plan lines 222 from a user overlaid on the displayed preview image 224, as depicted in FIGS. 5A and 6A.

At step 714 the processing and display device 220 determines which diode(s) of the plurality of laser diodes 202 to activate to produce guiding laser beam projections 226 corresponding to the one or more plan lines 222. In particular, the processing and display device 220 determines to direct guiding laser beam projections 226 which diode(s) of the plurality of laser diodes 202 to activate to produce the guiding laser beam projections 226 at the same location on the subject 228 in the real world as the one or more plan lines 222 overlaid on the preview image 224 of the subject 228.

At step 716 processing and display device 220 transmits a signal to activate the selected the laser diode(s) from step 714.

At step 718 the activated laser diode(s) directs one or more guiding laser beam projections 226 in the direction of the subject 228, as depicted in FIGS. 5B and 6B. The one or more guiding laser beam projections 226 create visible light lines on a surface of the subject 228. Additionally, the guiding laser beam projections 226 correspond to the location and angle of the one or more plan lines 222 created by the user on the preview image 224 as they related to the locations on the subject 228.

At step 720 the user (e.g., a surgeon) can being performing a fluoroscopic procedure based on the guiding laser beam projections 226 displayed on the subject 228. As a result of the process 700, the apparatus 200 provides improved surgical precision while significantly reducing dependence on intra-operative fluoroscopy (e.g., reducing exposure to radiation). By relying on the guiding laser beam projections 226 at different times during the fluoroscopic procedure instead of an active real time preview image 224 created through the first and second imaging assemblies, the process reduces an amount of radiation exposure to the subject 228 and any medical professionals within a given proximity to the imaging apparatus 200.

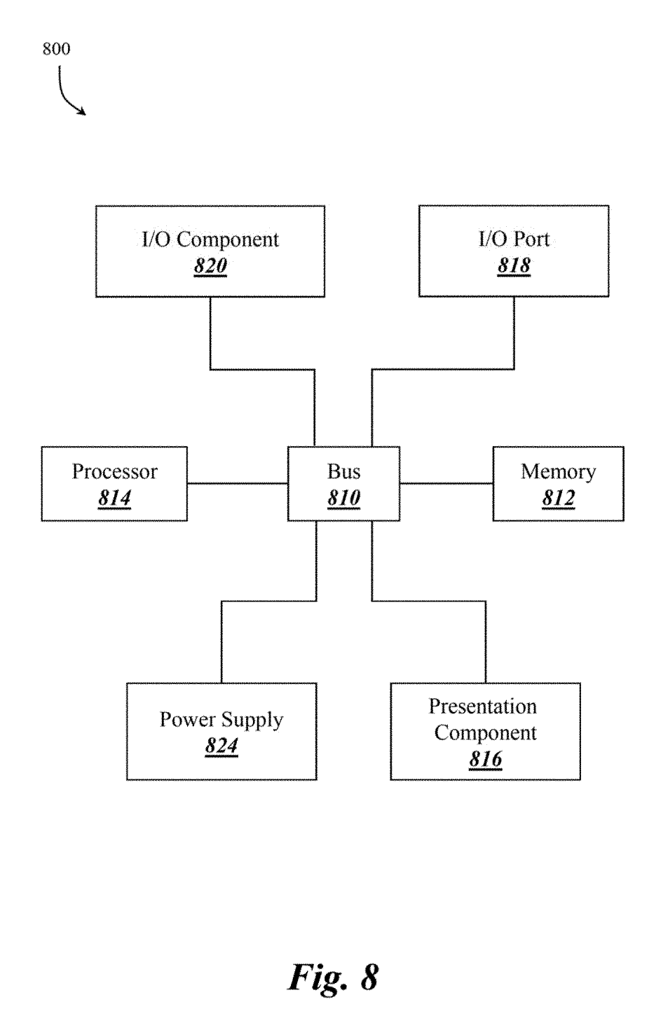

Any suitable computing device can be used to implement the computing devices 220 and methods/functionality described herein and be converted to a specific system for performing the operations and features described herein through modification of hardware, software, and firmware, in a manner significantly more than mere execution of software on a generic computing device, as would be appreciated by those of skill in the art. One illustrative example of such a computing device 800 is depicted in FIG. 8. The computing device 800 is merely an illustrative example of a suitable computing environment and in no way limits the scope of the present invention. A “computing device,” as represented by FIG. 8, can include a “workstation,” a “server,” a “laptop,” a “desktop,” a “hand-held device,” a “mobile device,” a “tablet computer,” or other computing devices, as would be understood by those of skill in the art. Given that the computing device 800 is depicted for illustrative purposes, embodiments of the present invention may utilize any number of computing devices 800 in any number of different ways to implement a single embodiment of the present invention. Accordingly, embodiments of the present invention are not limited to a single computing device 800, as would be appreciated by one with skill in the art, nor are they limited to a single type of implementation or configuration of the example computing device 800.

The computing device 800 can include a bus 810 that can be coupled to one or more of the following illustrative components, directly or indirectly: a memory 812, one or more processors 814, one or more presentation components 816, input/output ports 818, input/output components 820, and a power supply 824. One of skill in the art will appreciate that the bus 810 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, FIG. 8 is merely illustrative of an exemplary computing device that can be used to implement one or more embodiments of the present invention, and in no way limits the invention.

The computing device 800 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 800.

The memory 812 can include computer-storage media in the form of volatile and/or nonvolatile memory. The memory 812 may be removable, non-removable, or any combination thereof. Exemplary hardware devices are devices such as hard drives, solid-state memory, optical-disc drives, and the like. The computing device 800 can include one or more processors that read data from components such as the memory 812, the various I/O components 816, etc. Presentation component(s) 816 present data indications to a user or other device. Exemplary presentation components include a display device, speaker, printing component, vibrating component, etc.

The I/O ports 818 can enable the computing device 800 to be logically coupled to other devices, such as I/O components 820. Some of the I/O components 820 can be built into the computing device 800. Examples of such I/O components 820 include a microphone, joystick, recording device, game pad, satellite dish, scanner, printer, wireless device, networking device, and the like.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A medical imaging and guidance system, comprising:
a fluoroscopic imaging system, comprising:
a support gantry having a generally arc shape about an interior center focus point with a first terminal end and a second terminal end;
a first imaging assembly that is positioned on the support gantry and comprising a first imaging energy emitter that is positioned opposite a first imaging receptor, wherein one of the first imaging energy emitter or the first imaging receptor is positioned at the first terminal end of the support gantry; and
a plurality of laser-diodes fixedly attached to at least one of the first imaging receptor or the second imaging receptor around half of a circumference of the first imaging receptor or the second imaging receptor, respectively, at intervals of no greater than 15 radial degrees of spacing between each laser-diode of the plurality of laser-diodes;
wherein the plurality of laser-diodes emit guiding laser beam projections onto a subject to provide guidance to medical personnel during a procedure without radiation energy being emitted by the first imaging assembly.

2. The system of claim 1, further comprising:
a second imaging assembly that is positioned on the support gantry and comprising a second imaging energy emitter positioned that is opposite a second imaging receptor, wherein one of the second imaging energy emitter or the second imaging receptor is positioned at the second terminal end of the support gantry; and
a control unit that directs movement and positioning of the support gantry.

3. The system of claim 2, further comprising:
a processing and display device in communication with the first imaging assembly, the second imaging assembly, and the plurality of laser-diodes;
wherein the fluoroscopic imaging system obtains raw image data of a subject patient located proximate the interior center focus point between the first imaging assembly and the second imaging assembly and communicates the raw image data to the processing and display device;
wherein the processing and display device receives the raw image data and transforms the raw image data for display as a preview image and receives at least one plan line that is overlaid onto the preview image; and
wherein the processing and display device electronically selects one or more of the plurality of laser-diodes to activate to project one or more guiding laser beam projections onto the subject patient at locations corresponding to the at least one plan line.

4. The system of claim 3, wherein the plurality of laser-diodes are mechanically positioned with an angular coverage or linear translation around the fluoroscopic imaging system.

5. The system of claim 3, wherein the at least one plan line is generated with any position and line direction through the preview image.

6. The system of claim 5, wherein the at least one plan line provides input information and imaging geometry to determine power state of each of the plurality of laser-diodes.

7. The system of claim 2, wherein the first imaging assembly is positioned and oriented to emit imaging energy in an LT plane and the second imaging assembly is positioned and oriented to emit imaging energy in an AP plane, perpendicular to the LT plane.

8. The system of claim 2, wherein the first imaging assembly is positioned and oriented to emit imaging energy in an AP plane and the second imaging assembly is positioned and oriented to emit imaging energy in an LT plane, perpendicular to the AP plane.

9. The system of claim 2, wherein the first imaging receptor and the second imaging receptor are one of an image intensifier, a flat panel detector, or a thin film transistor (TFT) flat-panel detector with a scintillation material layer configured to readout a voltage data value to the processing and display device.

10. The system of claim 9, wherein the TFT flat-panel detector is configured to receive energy from visible photons that charge capacitors of pixel cells within the TFT flat-panel detector and charges from each of the pixel cells are readout as a voltage data value to the processing and display device.

11. The system of claim 2, wherein the first imaging energy emitter and the second imaging energy emitter are X-ray sources configured to produce X-ray beams.

12. The system of claim 1, wherein the plurality of laser-diodes is mechanically aligned to produce guiding laser beam projections to pass through the interior center focus point of the support gantry.

13. The system of claim 1, wherein the plurality of laser-diodes further comprise at least three laser-diodes uniformly spaced around the half of the circumference of the first imaging receptor or the second imaging receptor.

14. The system of claim 1, wherein an angle of convergence for each the plurality of laser-diodes is provided to focus each of the plurality of laser-diodes to a center of a front input plane of an imaging receptor that the plurality of laser-diodes is attached thereto.

15. The system of claim 1, each of the plurality of laser-diodes is independently operable according to a user specification.

16. A method for utilization of a medical procedure guidance system, the method comprising:
activating an imaging device comprising a first imaging assembly and a second imaging assembly configured and arranged to receive a subject patient therebetween;
obtaining, by imaging receptors, a raw image data of the subject patient;
communicating the raw image data to a processing and display device;
transforming the raw image data, by the processing and display device, into a preview image of the subject patient;
displaying the preview image on a display with at least one plan line, generated by the processing and display device, overlaid onto the preview image; and
instructing, by the processing and display device, one or more laser-diode of a plurality of laser-diodes in a laser-diode array to project one or more guiding laser beam projections onto the subject patient at locations corresponding to the at least one plan line, which is overlaid onto the preview image.

17. The method of claim 16, wherein the at least one plan line is generated in response to receiving user input to create the at least one plan line at a particular orientation and location on the preview image of the subject patient.

18. The method of claim 16, further comprising determining a positioning of the one or more laser-diode of the plurality of laser-diodes to generate the one or more guiding laser beam proj ections.

19. The method of claim 16, further comprising performing a fluoroscopic procedure relying on the one or more guiding laser beam projections.

20. The method of claim 16, wherein the imaging device further comprises:
a first imaging energy emitter, which is positioned opposite a first imaging receptor, wherein one of the first imaging energy emitter or the first imaging receptor is positioned at a first terminal end of a support gantry;
the second imaging assembly positioned on the support gantry, the second imaging assembly comprising a second imaging energy emitter positioned opposite a second imaging receptor, wherein one of the second imaging energy emitter or the second imaging receptor is positioned at a second terminal end of the support gantry;
a control unit that directs movement and positioning of the support gantry; and
the plurality of laser-diodes being fixedly attached to at least one of the first imaging receptor or the second imaging receptor around half of a circumference of the first imaging receptor or the second imaging receptor.
